SEQUENCE LISTING (16 sequences)

SEQ ID NO: 1 - DNA, 33 bp, Sorghum bicolor
ccggccgacctccactttcgtctctcccaatag

SEQ ID NO: 2 - DNA, 33 bp, Sorghum bicolor
ccggccgacctccactttcgtgtctcccaatag

SEQ ID NO: 3 - DNA, 33 bp, Sorghum bicolor
ccggcccagacaggacaggaggaggcctgtatc

SEQ ID NO: 4 - DNA, 33 bp, Sorghum bicolor
ccggcccagacaggaggaggcctgtatctgtat

SEQ ID NO: 5 - DNA, 33 bp, Sorghum bicolor
ccggccgtttttaaaggtggggttctccatgtt

SEQ ID NO: 6 - DNA, 33 bp, Sorghum bicolor
ccggccgtttttaaagatggggttctccatgtt

SEQ ID NO: 7 - DNA, 33 bp, Sorghum bicolor
ccggccacgcttttttaggtgggacccacggtc

SEQ ID NO: 8 - DNA, 33 bp, Sorghum bicolor
ccggccatgcttttctaggtgggacccatggtc

SEQ ID NO: 9 - DNA, 33 bp, Sorghum bicolor
ccggccgatgaatacagcgtcggccggggcgga

SEQ ID NO: 10 - DNA, 33 bp, Sorghum bicolor
ccggccgatgaatacagcgtcggccgggacgga

SEQ ID NO: 11 - DNA, 33 bp, Sorghum bicolor
ccggcctcgggctgcacggccagctagcatccg

SEQ ID NO: 12 - DNA, 33 bp, Sorghum bicolor
ccggcctctggctgcacggccagctagcatccg

SEQ ID NO: 13 - DNA, 33 bp, Sorghum bicolor
ccggcctagggcacgacatcactggcttccatt

SEQ ID NO: 14 - DNA, 33 bp, Sorghum bicolor
ccggcctaggcacgacatcactgccttccatta

SEQ ID NO: 15 - DNA, 33 bp, Sorghum bicolor
ccggcccacccaaccttcccatttgtcgatagc

SEQ ID NO: 16 - DNA, 33 bp, Sorghum bicolor
ccggcccacccaaccttcccatttcttctcttc

(19) United States
(12) Patent Application Publication
(10) Pub. No.: US 2017/0150693 A1
(43) Pub. Date: Jun. 1, 2017

(54) METHODS AND COMPOSITIONS FOR PRODUCING SORGHUM PLANTS WITH ANTHRACNOSE RESISTANCE

(71) Applicant: The Texas A&M University System, College Station, TX (US)

(72) Inventors: Patricia Klein, College Station, TX (US); William L. Rooney, College Station, TX (US); Anna Mildred Burrell, College Station, TX (US); Delroy Collins, College Station, TX (US)

(73) Assignee: The Texas A&M University System, College Station, TX (US)

(21) Appl. No.: 15/319,701

(22) PCT Filed: Jun. 17, 2015
(86) PCT No.: PCT/US2015/036173

§ 371 Date: Dec. 16, 2016

Related U.S. Application Data
(60) Provisional application No. 62/013,473, filed on Jun. 17, 2014.

(51) Int. Cl. A01H 5/10; A01H 1/04; A01H 1/02; C12Q 1/68

(52) U.S. Cl. CPC A01H 5/10; C12Q 1/6895; A01H 1/04; A01H 1/02; C12Q 2600/13; C12Q 2600/156

(57) ABSTRACT

The present disclosure provides unique sorghum plants with anthracnose resistance and their progeny. Such plants may comprise an introgressed QTL associated with anthracnose resistance. In certain aspects, compositions, including distinct polymorphic molecular markers, and methods for producing, breeding, identifying, selecting, and the like of plants or germplasm with anthracnose resistance are provided.

### CROSS REFERENCE TO RELATED APPLICATIONS

This application claims priority to U.S. Provisional Application No. 62/013,473, filed June 17, 2014, which is herein incorporated by reference in its entirety.

### FIELD OF THE INVENTION

The present invention relates to the field of plant breeding and, more specifically, to methods and compositions for producing sorghum plants with anthracnose resistance.

### INCORPORATION OF SEQUENCE LISTING

The sequence listing that is contained in the file named “TAMC029WO_ST25.txt,” which is 2.63 kilobytes as measured in Microsoft Windows operating system and was created on June 17, 2015, is filed electronically herewith and incorporated herein by reference.

### BACKGROUND OF THE INVENTION

Advances in molecular genetics have made it possible to select plants based on genetic markers linked to traits of interest, a process called marker-assisted selection (MAS). While breeding efforts to date have provided a number of useful sorghum lines and varieties with beneficial traits, there remains a need in the art for selection of varieties with further improved traits and methods for their production. In many cases, such efforts have been hampered by difficulties in identifying and using alleles conferring beneficial traits. These efforts can be confounded by the lack of definitive phenotypic assays, and other issues such as epistasis and polygenic or quantitative inheritance. In the absence of molecular tools such as MAS, it may not be practical to produce certain new genotypes of crop plants due to such challenges.

### SUMMARY OF THE INVENTION

In one aspect, the invention provides a sorghum plant comprising in its genome at least one introgressed allele locus associated with anthracnose resistance wherein the locus is in or genetically linked to a genomic region defined by loci c5_F_1666 (SEQ ID NOs:1 and 2) and c5_B_1937 (SEQ ID NOs:3 and 4) on sorghum chromosome 5, or within 15 cM thereof, or a progeny plant therefrom. In one embodiment, a locus associated with anthracnose resistance described herein is in a genomic region flanked by: loci c5_F_1666 (SEQ ID NOs:1 and 2) and c5_F_1893 (SEQ ID NOs:5 and 6) on sorghum chromosome 5; c5_F_1888 (SEQ ID NOs:13 and 14) and c5_F_1893 (SEQ ID NOs:5 and 6) on sorghum chromosome 5; or c5_F_1893 (SEQ ID NOs:5 and 6) and c5_B_1937 (SEQ ID NOs:3 and 4) on sorghum chromosome 5; or within 15 centimorgans (cM) thereof, including within 12 cM, 10 cM, 8 cM, 5 cM, 2 cM, 1 cM and 0 cM thereof. In another embodiment, the allele locus comprises at least one polymorphic nucleic acid selected from the group consisting of SEQ ID NOs:1-16. In another embodiment, the introgressed allele locus is introgressed from sorghum genotype SC748-5. In still another embodiment, the invention provides a part of such a sorghum plant, further defined as pollen, an ovule, a leaf, an embryo, a root, a root tip, an anther, a flower, a fruit, a stem, a shoot, a seed, a protoplast, a cell, and a callus.

In another aspect, the invention provides a method of detecting in at least one sorghum plant a genotype associated with anthracnose resistance, the method comprising the step of: (i) detecting in at least one sorghum plant an allele of at least one polymorphic nucleic acid that is associated with anthracnose resistance, wherein the polymorphic nucleic acid is in or genetically linked to a genomic region flanked by loci c5_F_1666 (SEQ ID NOs:1 and 2) and c5_B_1937 (SEQ ID NOs:3 and 4) on sorghum chromosome 5, or within 15 cM thereof. In one embodiment, the method further comprises the step of: (ii) identifying at least one sorghum plant in which a genotype associated with anthracnose resistance has been detected and denoting that the sorghum plant comprises a genotype associated with anthracnose resistance, or further comprises the step of: (iii) selecting a denoted sorghum plant from a population of plants. In another embodiment, the polymorphic nucleic acid is located in or genetically linked to a genomic region flanked by: loci c5_F_1666 (SEQ ID NOs:1 and 2) and c5_F_1893 (SEQ ID NOs:5 and 6) on sorghum chromosome 5; or c5_F_1893 (SEQ ID NOs:5 and 6) and c5_B_1937 (SEQ ID NOs:3 and 4) on sorghum chromosome 5; or within 15 cM thereof.

In another embodiment, at least one of said polymorphic nucleic acid is selected from the group consisting of SEQ ID NOs:1-16. In still another embodiment, the allele is introgressed from sorghum genotype SC748-5. In further embodiments, the invention provides a plant produced from such a method, or a seed that produces such a plant.

In another aspect, the invention provides a method for producing a sorghum plant that comprises in its genome at least one introgressed locus associated with anthracnose resistance, the method comprising: (i) crossing a first sorghum plant lacking a locus associated with anthracnose resistance with a second sorghum plant comprising a locus associated with anthracnose resistance located in a genomic region defined by loci c5_F_1666 (SEQ ID NOs:1 and 2) and c5_B_1937 (SEQ ID NOs:3 and 4) on sorghum chromosome 5, or within 15 cM thereof; (ii) detecting in progeny resulting from said crossing at least a first polymorphic nucleic acid in or genetically linked to said locus associated with anthracnose resistance; and (iii) selecting a sorghum plant comprising said polymorphic locus and said locus associated with anthracnose resistance. In one embodiment, the method further comprises the step of: (iv) crossing the sorghum plant of step (iii) with itself or another sorghum plant to produce a further generation. In another embodiment, steps (iii) and (iv) are repeated from about 3 times to about 10 times. In still another embodiment, the polymorphic nucleic acid is located in or genetically linked to a genomic region flanked by: loci c5_F_1666 (SEQ ID NOs:1 and 2) and c5_F_1893 (SEQ ID NOs:5 and 6) on sorghum chromosome 5; or c5_F_1893 (SEQ ID NOs:5 and 6) and c5_B_1937 (SEQ ID NOs:3 and 4) on sorghum chromosome 5; or within 15 cM thereof. In another embodiment, the invention provides a sorghum plant produced by such a method, or a progeny plant therefrom that comprises the introgressed locus associated with anthracnose resistance.

In another aspect, the invention provides a method of sorghum plant breeding, the method comprising the steps of: (i) selecting at least a first sorghum plant comprising at least one allele of a polymorphic nucleic acid that is genetically linked to a QTL associated with anthracnose resistance, wherein the QTL maps to a position between loci c5_F_1666 (SEQ ID NOs:1 and 2) and c5_B_1937 (SEQ ID NOs:3 and 4) on sorghum chromosome 5, or within 15 cM thereof; and (ii) crossing the first sorghum plant with itself or a second sorghum plant to produce progeny sorghum plants comprising the QTL associated with anthracnose resistance. In one embodiment, the QTL maps to a position between: loci c5_F_1666 (SEQ ID NOs:1 and 2) and c5_F_1893 (SEQ ID NOs:5 and 6) on sorghum chromosome 5; or c5_F_1893 (SEQ ID NOs:5 and 6) and c5_B_1937 (SEQ ID NOs:3 and 4) on sorghum chromosome 5; or within 15 cM thereof. In another embodiment, at least one of said polymorphic nucleic acid that is genetically linked to a QTL associated with anthracnose resistance is selected from the group consisting of SEQ ID NOs:1-16. In another embodiment, at least one polymorphic nucleic acid that is genetically linked to a QTL associated with anthracnose resistance maps within 40 cM, 20 cM, 15 cM, 10 cM, 5 cM, or 1 cM of the QTL associated with anthracnose resistance.

In still another aspect, the invention provides a method of introgressing an allele into a sorghum plant, the method comprising: (i) genotyping at least one sorghum plant in a population with respect to at least one polymorphic nucleic acid located in or genetically linked to a genomic region defined by loci c5_F_1666 (SEQ ID NOs:1 and 2) and c5_B_1937 (SEQ ID NOs:3 and 4) on sorghum chromosome 5, or within 15 cM thereof; (ii) selecting from the population at least one sorghum plant comprising at least one allele associated with anthracnose resistance. In one embodiment, the polymorphic nucleic acid is located in a genomic region flanked by: loci c5_F_1666 (SEQ ID NOs:1 and 2) and c5_F_1893 (SEQ ID NOs:5 and 6) on sorghum chromosome 5; or c5_F_1893 (SEQ ID NOs:5 and 6) and c5_B_1937 (SEQ ID NOs:3 and 4) on sorghum chromosome 5; or within 15 cM thereof. In another embodiment, at least one of said polymorphic nucleic acid is selected from the group consisting of SEQ ID NOs:1-16. In another embodiment, the invention provides a sorghum plant obtained by such a method.

### BRIEF DESCRIPTION OF THE DRAWING

FIG. 1a and FIG. 1b-Linkage map of 10 sorghum chromosomes created using 117 F₅ recombinant inbred lines (RILs) from a cross between BTx623 and SC748-5. The map contains 619 SNP and 3 SSR markers and spans 1269.9 cM.

### BRIEF DESCRIPTION OF THE SEQUENCES

SEQ ID NO:1 -DNA sequence of marker c5_F_1666 from sorghum variety BTx623.
SEQ ID NO:2 -DNA sequence of marker c5_F_1666 from sorghum variety SC748-5.
SEQ ID NO:3 -DNA sequence of marker c5_B_1937 from sorghum variety BTx623.
SEQ ID NO:4 -DNA sequence of marker c5_B_1937 from sorghum variety SC748-5.
SEQ ID NO:5 -DNA sequence of marker c5_F_1893 from sorghum variety BTx623.
SEQ ID NO:6 -DNA sequence of marker c5_F_1893 from sorghum variety SC748-5.
SEQ ID NO:7 -DNA sequence of marker c5_B_1853 from sorghum variety BTx623.
SEQ ID NO:8 -DNA sequence of marker c5_B_1853 from sorghum variety SC748-5.
SEQ ID NO:9 -DNA sequence of marker c5_F_1867 from sorghum variety BTx623.
SEQ ID NO:10 -DNA sequence of marker c5_F_1867 from sorghum variety SC748-5.
SEQ ID NO:11 -DNA sequence of marker c5_F_1870 from sorghum variety BTx623.
SEQ ID NO:12 -DNA sequence of marker c5_F_1870 from sorghum variety SC748-5.
SEQ ID NO:13 -DNA sequence of marker c5_F_1888 from sorghum variety BTx623.
SEQ ID NO:14 -DNA sequence of marker c5_F_1888 from sorghum variety SC748-5.
SEQ ID NO:15 -DNA sequence of marker c5_B_1938 from sorghum variety BTx623.
SEQ ID NO:16 -DNA sequence of marker c5_B_1938 from sorghum variety SC748-5.

### DETAILED DESCRIPTION OF THE INVENTION

The invention provides methods and compositions for producing sorghum plants with anthracnose resistance through introgression of a major QTL described herein. In accordance with the invention the introgressed locus allele may be newly introgressed into a given desired genomic background of a specific sorghum variety or cultivar. The anthracnose resistance allele as described herein may be introgressed from a particular resistant sorghum line, such as sorghum genotype SC748-5. In an embodiment, a QTL for anthracnose resistance may be introgressed from any sorghum plant comprising anthracnose resistance. Certain embodiments of the invention provide methods of detecting in a sorghum plant a genotype associated with anthracnose resistance. Other embodiments provide methods of identifying and selecting a sorghum plant comprising in its genome a genotype associated with anthracnose resistance. In other embodiments, methods of producing a sorghum plant that comprises in its genome at least one introgressed locus associated with anthracnose resistance and methods for introgressing such an allele into a sorghum plant are provided. Sorghum plants and parts thereof made by any of said methods are also provided for, as well as polymorphic nucleic acid sequences that may be used in the production and identification of such plants.

Interest in sorghum as a potential fossil fuel alternative has risen in recent years due to its low cost production, use of conventional harvest equipment, and its ability to grow on marginal soils considered unsuitable for food crop production. Anthracnose infection in sorghum can cause lower yields of grain and biomass and in some cases, complete crop failure and thus, development of sorghum varieties with resistance to anthracnose has significance to growers, processors, retailers, and customers. Colletotrichum sublineolum, the causal agent of anthracnose, is a major disease of sorghum capable of infecting all parts of the plant and causing significant economic loss. Anthracnose is particularly problematic in tropical and sub-tropical regions with high humidity and heat, where reduction in yield can range from 20 to 80%. The pathogen infects most sorghum species, particularly Sorghum halepense (L.) Pers. (Johnsongrass). Host plant resistance to Colletotrichum sublineolum remains the most effective means for controlling anthracnose.

Numerous sources of genetic resistance to anthracnose have been identified but consistency of resistance is a function of not only specific resistance sources but also the pathotype and environment of evaluation. Sorghum line SC748-5 has been identified as strongly resistant to the anthracnose disease and has maintained its resistance over many years and different environmental conditions, a unique trait, as most anthracnose resistant sources in sorghum are environment and disease pathotype specific. Anthracnose resistance in SC748-5 has been reported to be controlled by a single dominant locus. A genetic locus, Cgl, was also identified at the distal end of Linkage Group J, now identified as Sorghum bicolor chromosome 5 through physical mapping and genome sequencing.

To determine the genetic mechanism of anthracnose resistance, the inventors crossed resistant sorghum line SC748-5 with a susceptible line, BTx623. A recombinant inbred line (RIL) population was created for genotyping, phenotyping, and identifying genes conferring resistance to anthracnose in sorghum. As reported herein, the current inventors have identified for the first time SNP markers delimiting a major QTL for anthracnose resistance in sorghum genotype SC748-5 that explained up to 40% of the phenotypic variation. This QTL allows for production and development of new or improved sorghum varieties with anthracnose resistance.

SNP markers in the proximity of this QTL conferring anthracnose resistance were identified, and may be used in marker-assisted breeding programs to introgress the QTL conferring anthracnose resistance derived from the SC748-5 parent into other sorghum lines or desirable germplasm to produce new sorghum lines with anthracnose resistance, such as by marker-assisted selection and/or marker-assisted backcrossing. Additionally, resequencing of the anthracnose resistant parent, SC748-5, identified numerous amino acid changes in disease resistance genes located within the anthracnose QTL, suggesting that resistance may be controlled by a group of disease resistance genes.

Certain embodiments of the present invention thus provide sorghum plants comprising in their genome at least a first introgressed locus conferring anthracnose resistance. In accordance with the invention, the introgressed locus allele may not previously have been introgressed into the given genomic background of the specific variety or cultivar developed. Certain embodiments provide for methods of detecting in a sorghum plant a genotype associated with anthracnose resistance. Certain embodiments also provide methods of identifying and selecting a sorghum plant comprising in its genome a genotype associated with anthracnose resistance. Further, certain embodiments provide methods of producing a sorghum plant that comprises in its genome at least one introgressed locus associated with anthracnose resistance and methods for introgressing such an allele into a sorghum plant. Sorghum plants and parts thereof made by any of said methods are also provided for in certain embodiments of the invention as well as polymorphic nucleic acid sequences that may be used in the production and identification of such plants.

By providing markers to confer anthracnose resistance, the invention results in significant economization by substituting costly and time-intensive phenotyping assays with genotyping. Further, breeding programs can be designed to explicitly drive the frequency of specific favorable phenotypes by targeting particular genotypes. Fidelity of these associations may be monitored continuously to ensure maintained predictive ability and, thus, informed breeding decisions.

In accordance with the invention, one of skill in the art may thus identify a candidate germplasm source possessing anthracnose resistance as described herein, but which is lacking one or more traits which the plant breeder seeks to have in a variety or parent line thereof. The techniques of the invention may thus be used to identify desirable phenotypes by identifying genetic markers associated with the phenotype, or such techniques may employ phenotypic assays to identify desired plants either alone or in combination with genetic assays, thereby also identifying a marker genotype associated with the trait that may be used for production of new varieties with the methods described herein.

The invention thus provides for the introgression of at least a first locus conferring anthracnose resistance into a given genetic background. Successful sorghum production depends on attention to various horticultural practices. These include soil management with special attention to proper fertilization, crop establishment with appropriate spacing, weed control, the introduction of bees or other insects for pollination, irrigation, and pest management.

Sorghum crops can be established from seed or from transplants. Transplanting can result in an earlier crop compared with a crop produced from direct seeding. When a grower wants to raise a seedless fruited crop, transplanting can be preferred. Transplanting helps achieve complete plant stands rapidly, especially where higher seed costs make direct-seeding risky.

Sorghum breeders are challenged with anticipating changes in growing conditions, new pathogen pressure, and changing consumer preferences. With these projections, a breeder will attempt to create new cultivars that will fit the developing needs of growers, shippers, retailers, and consumers. Thus, the breeder is challenged to combine in a single genotype as many favorable attributes as possible for good growing distribution and eating.

### Development of Sorghum Varieties With Anthracnose Resistance

Anthracnose infection in sorghum can cause lower yields of grain and biomass and in some cases, complete crop failure and thus, development of sorghum varieties with resistance to anthracnose has significance to growers, processors, retailers, and customers. The current inventors have identified for the first time SNP markers delimiting a major QTL for anthracnose resistance in sorghum genotype SC748-5 that allows for production and development of new or improved sorghum varieties with anthracnose resistance, as well as single nucleotide polymorphism (SNP) markers in the proximity of this locus that can be used for the tracking and introgression of this genomic region to desirable germplasm, such as by marker-assisted selection and/or marker-assisted backcrossing. As reported herein, a recombinant inbred line population (RIL) was developed from a cross between sorghum lines BTx623 (susceptible) and SC748-5 (resistant). A linkage map was constructed with 619 single nucleotide polymorphism (SNP) markers and three microsatellites and a total map length of 1269.9 cM. QTL mapping analysis in the two populations identified a QTL on sorghum chromosome 5 for anthracnose resistance.

The invention thus contemplates the tracking and introduction of QTL conferring anthracnose resistance into a given genetic background. One of ordinary skill will understand that anthracnose resistance may be introgressed from one genotype to another using a primary locus described herein via marker-assisted selection. Accordingly, a germplasm source can be selected that has anthracnose resistance, and/or additional desired phenotypes or traits. In an embodiment, a sorghum plant in accordance with the invention may be from any sorghum species or genotype. A breeder may now select anthracnose resistance during breeding using marker-assisted selection for the region described herein. Provided with the present disclosure, one of ordinary skill can introduce anthracnose resistance into any genetic background.

Thus, the QTL identified herein on sorghum chromosome 5 may be used for marker-assisted selection for anthracnose resistance in sorghum. This discovery of QTL conferring anthracnose resistance may facilitate the development of sorghum plants or lines having anthracnose resistance.

For most breeding objectives, commercial breeders work within germplasm that is often referred to as the “cultivated type.” This germplasm is easier to breed with because it generally performs well when evaluated for horticultural performance. The performance advantage the cultivated type provides is sometimes offset by a lack of allelic diversity. This is the tradeoff a breeder accepts when working with cultivated germplasm—better overall performance, but a lack of allelic diversity. Breeders generally accept this tradeoff because progress is faster when working with cultivated material than when breeding with genetically diverse sources.

In contrast, when a breeder makes either intra-specific crosses, or inter-specific crosses, a converse trade off occurs. In these examples, a breeder typically crosses cultivated germplasm with a non-cultivated type. In such crosses, the breeder can gain access to novel alleles from the non-cultivated type, but may have to overcome the genetic drag associated with the donor parent. Because of the difficulty with this breeding strategy, this approach often fails because of fertility and fecundity problems. The difficulty with this breeding approach extends to many crops, and is exemplified with an important disease resistant phenotype that was first described in tomato in 1944 (Smith, Proc. Am. Soc. Hort. Sci. 44:413-16). In this cross, a nematode disease resistance was transferred from L. peruvianum (PI128657) into a cultivated tomato. Despite intensive breeding, it was not until the mid-1970's before breeders could overcome the genetic drag and release successful lines carrying this trait. Indeed, even today, tomato breeders deliver this disease resistance gene to a hybrid variety from only one parent. This allows the remaining genetic drag to be masked. The inventiveness of succeeding in this breeding approach has been recognized by the USPTO (U.S. Pat. Nos.: 6,414,226, 6,096,944, 5,866,764, and 6,639,132).

Some phenotypes are determined by the genotype at one locus. These simple traits, like those studied by Gregor Mendel, fall in discontinuous categories such as green or yellow seeds. Most variation observed in nature, however, is continuous, like yield in field corn, or human blood pressure. Unlike simply inherited traits, continuous variation can be the result of polygenic inheritance. Loci that affect continuous variation are referred to as quantitative trait loci (QTLs). Variation in the phenotype of a quantitative trait is the result of the allelic composition at the QTLs and the environmental effect. The heritability of a trait is the proportion of the phenotypic variation attributed to the genetic variance. This ratio varies between 0 and 1.0. Thus, a trait with heritability near 1.0 is not greatly affected by the environment. Those skilled in the art recognize the importance of creating commercial lines with high heritability horticultural traits because these cultivars will allow growers to produce a crop with uniform market specifications.

### Genomic Region, QTL, Polymorphic Nucleic Acids, and Alleles Associated With Sorghum Anthracnose Resistance

Using recombinant inbred line (RIL) F₅ plants derived from a cross of sorghum lines BTx623 (susceptible) and SC748-5 (resistant), the inventors have discovered a genomic region, QTL, alleles, polymorphic nucleic acids, linked markers, and the like that when present in certain allelic forms are associated with sorghum anthracnose resistance.

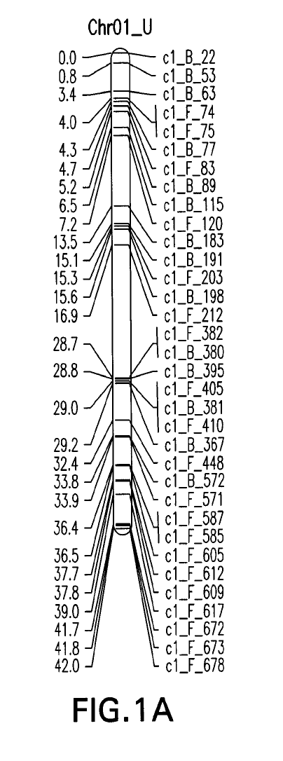

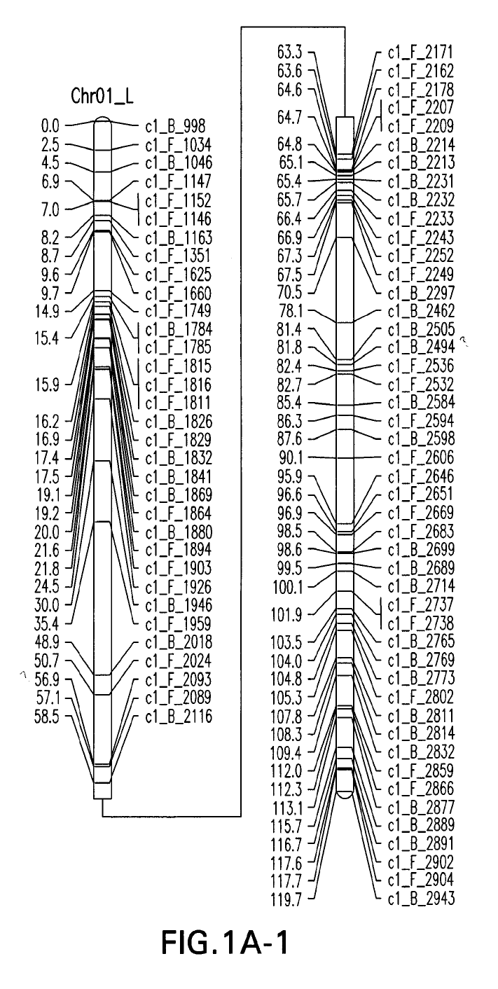

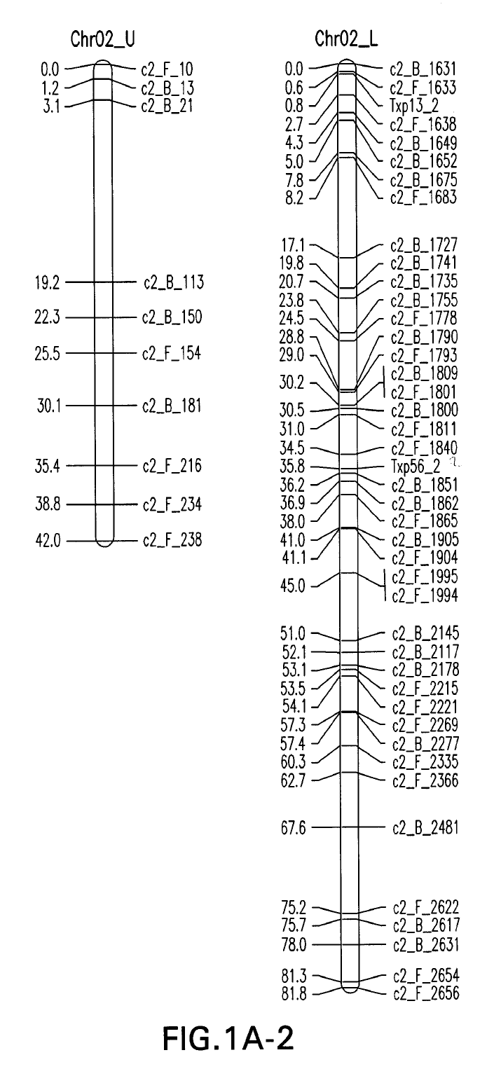

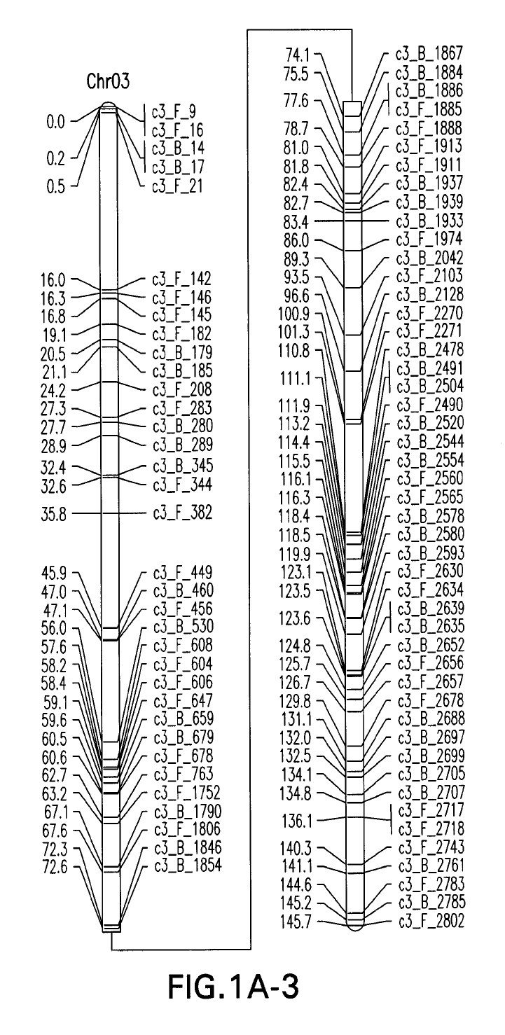

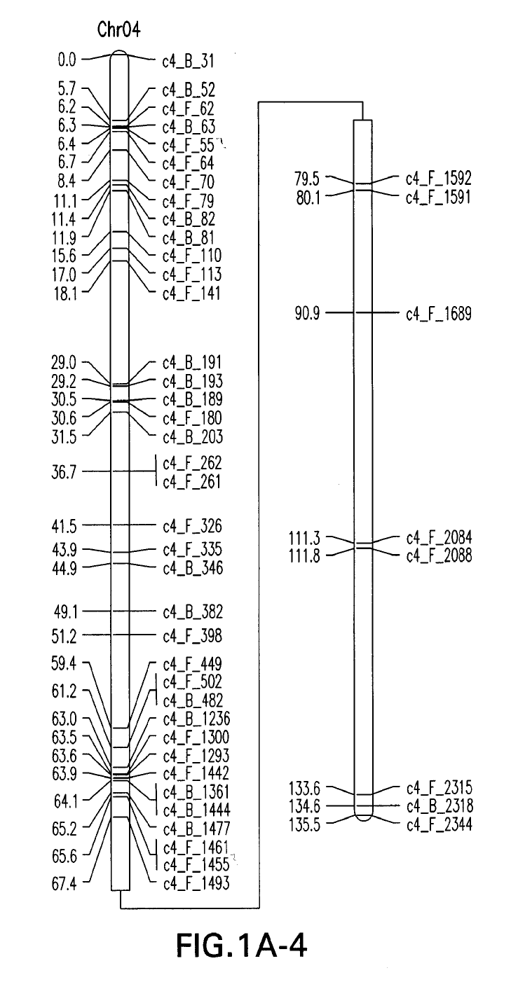

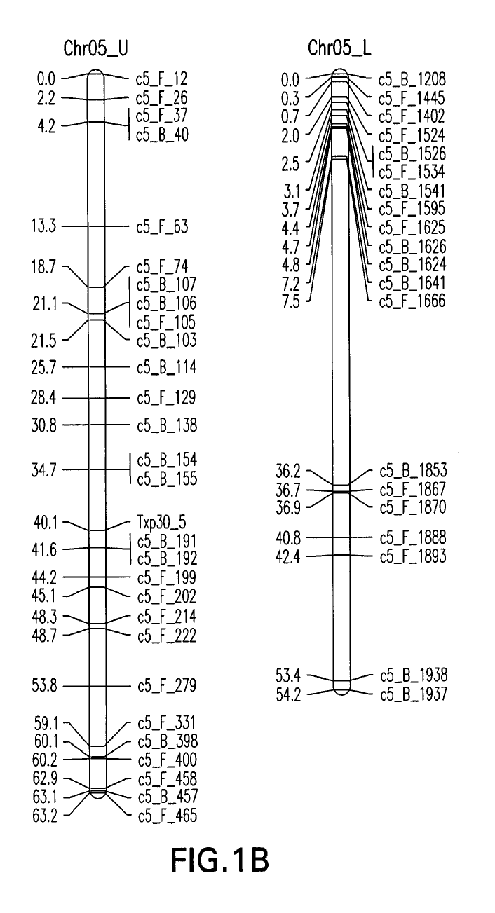

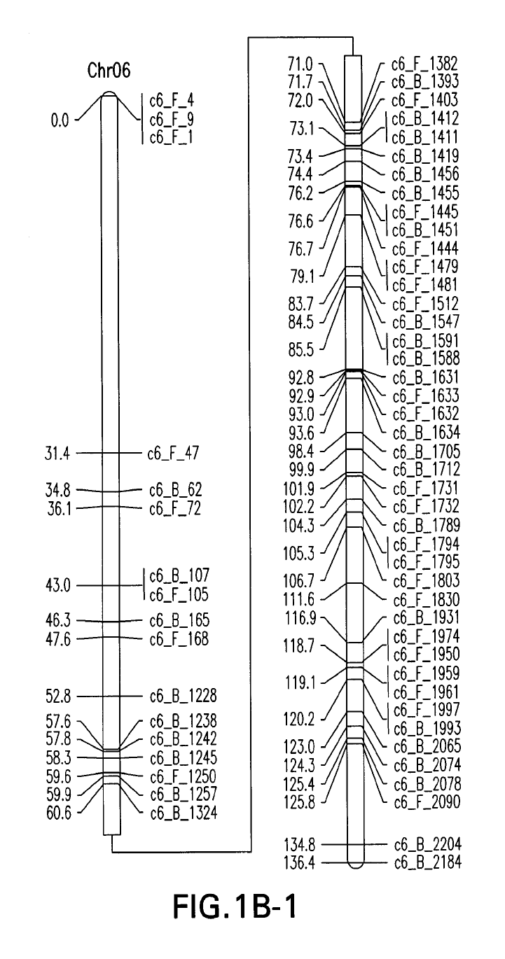

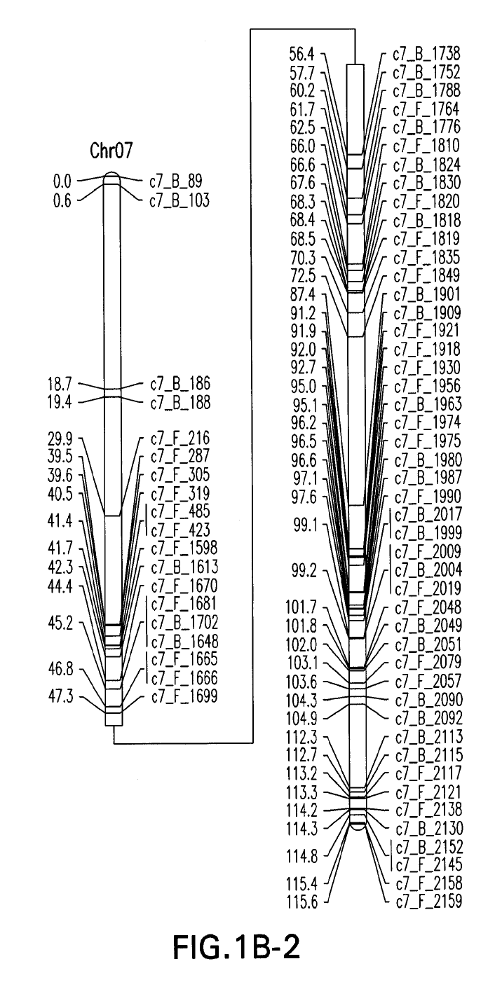

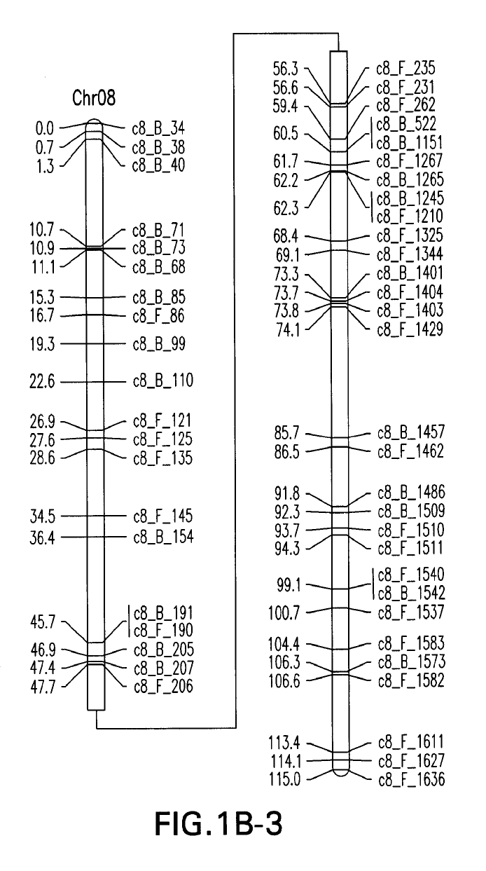

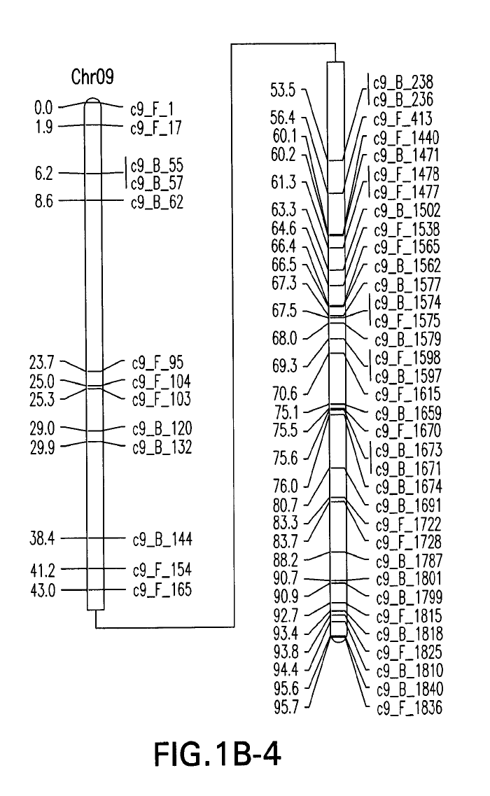

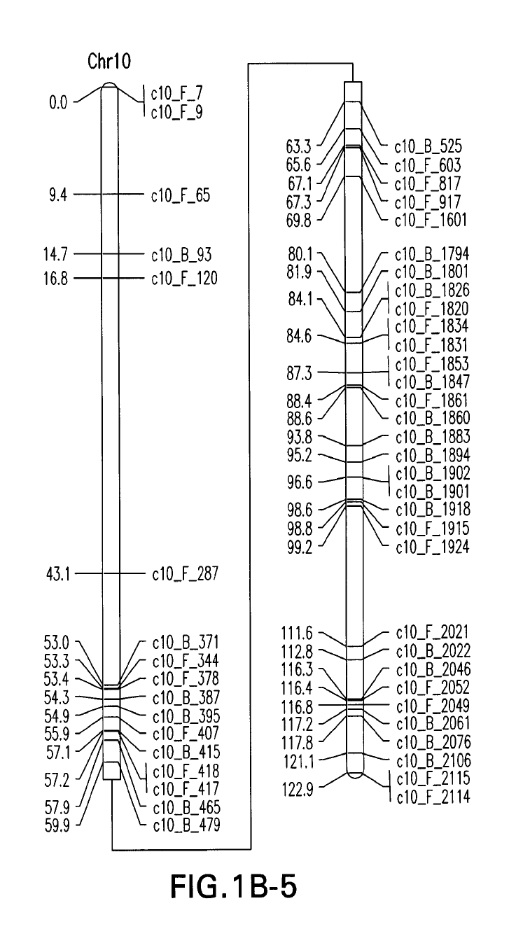

A major QTL associated with anthracnose resistance was identified on sorghum chromosome 5 and, on the genetic map provided herein as FIG. 1a and FIG. 1b, defined by loci c5_F_1666 (SEQ ID NOs:1 and 2) and c5_B_1937 (SEQ ID NOs:3 and 4). Certain of the various embodiments of the present disclosure utilize a QTL or polymorphic nucleic acid marker or allele located within this region or within one or more subregions on sorghum chromosome 5. For example, in an embodiment, a region or subregion of the major QTL for anthracnose resistance can be described as being defined by loci c5_F_1666 (SEQ ID NOs:1 and 2) and c5_F_1893 (SEQ ID NOs:5 and 6) on sorghum chromosome 5, or by c5_F_1893 (SEQ ID NOs:5 and 6) and c5_B_1937 (SEQ ID NOs:3 and 4) on sorghum chromosome 5. In another embodiment, a marker such as one set forth herein as SEQ ID NOs:1-16 may be used to describe or identify the QTL described herein conferring anthracnose resistance.

The above markers and allelic states are exemplary. One of skill in the art would recognize how to identify sorghum plants with other polymorphic nucleic acid markers and allelic states thereof related to sorghum anthracnose resistance consistent with the present disclosure. One of skill the art would also know how to identify the allelic state of other polymorphic nucleic acid markers located in the genomic region(s) or linked to the QTL or other markers identified herein, to determine their association with sorghum anthracnose resistance.

Certain embodiments of the invention contemplate the use of dihaploidization to produce an inbred line. A haploid plant has only one copy of each chromosome instead of the normal pair of chromosomes in a diploid plant. Haploid plants can be produced, for example, by treating with a haploid inducer. Haploid plants can be subjected to treatment that causes the single copy chromosome set to double, producing a duplicate copy of the original set. The resulting plant is termed a “double-haploid” and contains pairs of chromosomes that are generally in a homozygous allelic state at any given locus. Dihaploidization can reduce the time required to develop new inbred lines in comparison to developing lines through successive rounds of backcrossing.

One of skill in the art would understand that polymorphic nucleic acids that are located in the genomic regions identified herein may be used in certain embodiments of the methods of the invention. Given the provisions herein of a genomic region, QTL, and polymorphic markers identified herein, additional markers located either within or near a genomic region described herein that are associated with the phenotype can be obtained by typing new markers in various germplasm. The genomic region, QTL, and polymorphic markers identified herein can also be mapped relative to any publicly available physical or genetic map to place the region described herein on such map. One of skill in the art would also understand that additional polymorphic nucleic acids that are genetically linked to the QTL associated with anthracnose resistance and that map within 40 cM, 20 cM, 10 cM, 5 cM, or 1 cM of the QTL or the markers associated with anthracnose resistance may also be used.

Introgression of a Genomic Locus Associated with Anthracnose Resistance

Provided herein are unique sorghum germplasms or sorghum plants comprising an introgressed genomic region that is associated with anthracnose resistance and methods of obtaining the same. Marker-assisted introgression involves the transfer of a chromosomal region, defined by one or more markers, from one germplasm to a second germplasm. Offspring of a cross that contain the introgressed genomic region can be identified by the combination of markers characteristic of the desired introgressed genomic region from a first germplasm (e.g., anthracnose resistance germplasm) and both linked and unlinked markers characteristic of the desired genetic background of a second germplasm.

Flanking markers that identify a genomic region associated with anthracnose resistance can include any loci or markers described herein on sorghum chromosome 5; and those that identify sub-regions thereof include can include any loci or loci intervals described herein. In an embodiment, the QTL for anthracnose resistance as described herein may be described by one or more of the markers or loci set forth herein as SEQ ID NOs:1-16.

For example, markers that may define or be genetically linked to a genomic region or subregion include those defined by loci c5_F_1666 (SEQ ID NOs:1 and 2) and c5_B_1937 (SEQ ID NOs:3 and 4) on sorghum chromosome 5, or within 15 cM thereof. In further embodiments, markers are provided in a genomic region defined or genetically linked to loci c5_F_1666 (SEQ ID NOs:1 and 2) and c5_F_1893 (SEQ ID NOs:5 and 6) on sorghum chromosome 5, or by c5_F_1893 (SEQ ID NOs:5 and 6) and c5_B_1937 (SEQ ID NOs:3 and 4) on sorghum chromosome 5; or within 15 cM thereof. One of skill in the art will understand that other markers may be identified within this regions that may be useful in accordance with the invention.

Flanking markers that fall on both the telomere proximal end and the centromere proximal end of any of these genomic intervals may be useful in a variety of breeding efforts that include, but are not limited to, introgression of genomic regions associated with anthracnose resistance into a genetic background comprising markers associated with germplasm that ordinarily contains a genotype associated with another phenotype.

Markers that are linked and either immediately adjacent or adjacent to the identified anthracnose resistance QTL that permit introgression of the QTL in the absence of extraneous linked DNA from the source germplasm containing the QTL are provided herewith. Those of skill in the art will appreciate that when seeking to introgress a smaller genomic region comprising a QTL associated with anthracnose resistance described herein, that any of the telomere proximal or centromere proximal markers that are immediately adjacent to a larger genomic region comprising the QTL can be used to introgress that smaller genomic region.

A marker within about 40 cM of a marker of an anthracnose resistance QTL described herein may be useful in a variety of breeding efforts that include, but are not limited to, introgression of genomic regions associated with anthracnose resistance into a genetic background comprising markers associated with germplasm that ordinarily contains a genotype associated with another phenotype. For example, a marker within 40 cM, 20 cM, 15 cM, 10 cM, ScM, 2 cM, or 1 cM of an anthracnose resistance QTL or marker described herein can be used for marker-assisted introgression of anthracnose resistance.

Sorghum plants or germplasm comprising an introgressed region that is associated with anthracnose resistance wherein at least 10%, 25%, 50%, 75%, 90%, or 99% of the remaining genomic sequences carry markers characteristic of plant or germplasm that otherwise or ordinarily comprise a genomic region associated with another phenotype, are thus provided. Furthermore, sorghum plants comprising an introgressed region where closely linked regions adjacent and/or immediately adjacent to the genomic regions, QTL, and markers provided herewith that comprise genomic sequences carrying markers characteristic of sorghum plants or germplasm that otherwise or ordinarily comprise a genomic region associated with the phenotype are also provided.

### Molecular Assisted Breeding Techniques

A number of different marker types are available for use in genetic mapping and may be useful in accordance with the invention. Genetic markers that can be used in the practice of the present invention include, but are not limited to, Simple Sequence Repeats (SSR), Single Nucleotide Polymorphisms (SNP), Restriction Fragment Length Polymorphisms (RFLP), Amplified Fragment Length Polymorphisms (AFLP), Simple Sequence Length Polymorphisms (SSLPs), Insertion/Deletion Polymorphisms (Indels), Variable Number Tandem Repeats (VNTR), and Random Amplified Polymorphic DNA (RAPD), nucleotide insertions and/or deletions (INDELs), isozymes, and others known to those skilled in the art. Marker discovery and development in crops provides the initial framework for applications to marker-assisted breeding activities (U.S. Patent Pub. Nos.: 2005/0204780, 2005/0216545, 2005/0218305, and 2006/00504538). The resulting “genetic map” is the representation of the relative position of characterized loci (polymorphic nucleic acid markers or any other locus for which alleles can be identified) to each other.

Polymorphisms comprising as little as a single nucleotide change can be assayed in a number of ways. For example, detection can be made by electrophoretic techniques including a single strand conformational polymorphism (Orita et al. (1989) Genomics, 8(2), 271-278), denaturing gradient gel electrophoresis (Myers (1985) EPO 0273085), or cleavage fragment length polymorphisms, but the widespread availability of DNA sequencing machines often makes it easier to just sequence amplified products directly. Once the polymorphic sequence difference is known, rapid assays can be designed for progeny testing, typically involving some version of PCR amplification of specific alleles (PASA, Sommer, et al. (1992) Biotechniques 12(1), 82-87), or PCR amplification of multiple specific alleles (PAMSA, Dutton and Sommer (1991) Biotechniques, 11(6), 700-7002).

In accordance with the above, a single nucleotide polymorphism (SNP) genetic map has been produced using sorghum parent lines BTx623 (susceptible) and SC748-5 (resistant). Results described herein identify a major QTL on sorghum chromosome 5 that confers resistance to anthracnose in sorghum. As described further herein, this QTL can be a target for marker-assisted selection of anthracnose resistance in sorghum breeding programs.

As a set, polymorphic markers serve as a useful tool for fingerprinting plants to inform the degree of identity of lines or varieties (U.S. Pat. No. 6,207,367). These markers form the basis for determining associations with phenotypes and can be used to drive genetic gain. In certain embodiments of methods of the invention, polymorphic nucleic acids can be used to detect in a sorghum plant a genotype associated with anthracnose resistance, identify a sorghum plant with a genotype associated with anthracnose resistance, and to select a sorghum plant with a genotype associated with anthracnose resistance. In certain embodiments of methods of the invention, polymorphic nucleic acids can be used to produce a sorghum plant that comprises in its genome an introgressed locus associated with anthracnose resistance. In certain embodiments of the invention, polymorphic nucleic acids can be used to breed progeny sorghum plants comprising a locus associated with anthracnose resistance.

Certain genetic markers may include “dominant” or “codominant” markers. “Codominant” markers reveal the presence of two or more alleles (two per diploid individual). “Dominant” markers reveal the presence of only a single allele. Markers are preferably inherited in codominant fashion so that the presence of both alleles at a diploid locus, or multiple alleles in triploid or tetraploid loci, are readily detectable, and they are free of environmental variation, i.e., their heritability is 1. A marker genotype typically comprises two marker alleles at each locus in a diploid organism. The marker allelic composition of each locus can be either homozygous or heterozygous. Homozygosity is a condition where both alleles at a locus are characterized by the same nucleotide sequence. Heterozygosity refers to different conditions of the allele at a locus.

Nucleic acid-based analyses for determining the presence or absence of the genetic polymorphism (i.e., for genotyping) can be used in breeding programs for identification, selection, introgression, and the like. A wide variety of genetic markers for the analysis of genetic polymorphisms are available and known to those of skill in the art. The analysis may be used to select for genes, portions of genes, QTL, alleles, or genomic regions that comprise or are linked to a genetic marker that is linked to or associated with anthracnose resistance in sorghum.

As used herein, nucleic acid analysis methods include, but are not limited to, PCR-based detection methods (for example, TaqMan assays), microarray methods, mass spectrometry-based methods and/or nucleic acid sequencing methods, including whole genome sequencing. In certain embodiments, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. However, the compositions and methods of the present invention can be used in conjunction with any polymorphism typing method to type polymorphisms in genomic DNA samples. These genomic DNA samples used include but are not limited to genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequences can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is disclosed in U.S. Pat. Nos. 6,799,122; 6,913,879; and 6,996,476.

Target nucleic acid sequence can also be detected by probe linking methods as disclosed in U.S. Pat. No. 5,616,464, employing at least one pair of probes having sequences homologous to adjacent portions of the target nucleic acid sequence and having side chains which non-covalently bind to form a stem upon base pairing of the probes to the target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283. SBE methods are based on extension of a nucleotide primer that is adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of genomic DNA which flanks a region containing the polymorphism to be assayed. Following amplification of the region of the genome containing the polymorphism, the PCR product is mixed with the third oligonucleotide (called an extension primer) which is designed to hybridize to the amplified DNA adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, the locus or loci of interest can be directly sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays, as reviewed by R. F. Service Science 2006 311:1544-1546.

The markers to be used in the methods of the present invention should preferably be diagnostic of origin in order for inferences to be made about subsequent populations. Experience to date suggests that SNP markers may be ideal for mapping because the likelihood that a particular SNP allele is derived from independent origins in the extant populations of a particular species is very low. As such, SNP markers appear to be useful for tracking and assisting introgression of QTLs.

### Definitions

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “plant” includes plant cells, plant protoplasts, plant cells of tissue culture from which sorghum plants can be regenerated, plant calli, plant clumps and plant cells that are intact in plants or parts of plants such as pollen, flowers, seeds, leaves, stems, and the like. As used herein, “sorghum ” or “sorghum plant” refers to any plant of the Sorghum species.

As used herein, the term “population” means a genetically heterogeneous collection of plants that share a common parental derivation.

As used herein, the terms “variety,” “cultivar,” and “line” mean a group of similar plants that by their genetic pedigrees and performance can be identified from other varieties within the same species.

As used herein, an “allele” refers to one of two or more alternative forms of a genomic sequence at a given locus on a chromosome.

A “Quantitative Trait Locus (QTL)” is a chromosomal location that encodes for alleles that affect the expressivity of a phenotype.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of a plant.

As used herein, the term “introgressed,” when used in reference to a genetic locus, refers to a genetic locus that has been introduced into a new genetic background. Introgression of a genetic locus can thus be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the term “linked,” when used in the context of nucleic acid markers and/or genomic regions, means that the markers and/or genomic regions are located on the same linkage group or chromosome such that they tend to segregate together at meiosis.

As used herein, the term “denoting” when used in reference to a plant genotype refers to any method whereby a plant is indicated to have a certain genotype. This includes any means of identification of a plant having a certain genotype. Indication of a certain genotype may include, but is not limited to, any entry into any type of written or electronic medium or database whereby the plant's genotype is provided. Indications of a certain genotype may also include, but are not limited to, any method where a plant is physically marked or tagged. Illustrative examples of physical marking or tags useful in the invention include, but are not limited to, a barcode, a radio-frequency identification (RFID), a label, or the like.

### EXAMPLES

The following disclosed embodiments are merely representative of the invention which may be embodied in various forms. Thus, specific structural, functional, and procedural details disclosed in the following examples are not to be interpreted as limiting.

### Example 1

### Genotyping

A recombinant inbred line (RIL) population derived from the cross of BTx623 ×SC748-5 was developed as previously described (Mehta et al., Field Crops Research 93:1-9, 2005). The parental genotypes and 117 F₅ lines were genotyped, using a genotyping-by-sequencing methodology developed specifically for C4 grasses, known as Digital Genotyping (DG) (Morishige et al., BMC Genomics 14:448, 2013).

Seeds from each line were germinated in Sunshine MVP growing media (Sun Gro Horticulture) in a greenhouse for 14 days under natural sunlight supplemented with sodium halide lights. Temperatures varied from 24° C. (night) to 30° C. (day). Total genomic DNA was extracted from leaf tissue from 10-12 seedlings from each RIL or parental line using the FastPrep FP120 instrument (Bio 101, Savant). DNA was extracted using the FastDNA Spin Kit (MP Biomedicals) according to the manufacturer's protocol to obtain sequence-quality DNA. Purified genomic DNA was quantitated fluorometrically using a Qubit Fluorometer (Invitrogen).

DG template libraries were prepared by digesting 500 ng of each DNA with the methyl-sensitive enzyme, Fsel (New England Biolabs). Following digestion, multiplex identifier barcodes were ligated to the fragments, which were subsequently grouped into pools of 48 DNAs, each containing a unique 4-bp barcode. The pools were randomly sheared by sonication with a target size of 250 bp then size-selected on a 2% agarose gel to a range of 250 bp ±50 bp. Following overhang fill-in, blunting, and adenylation, the pools underwent ligation of an Illumina-specific adapter and were purified using magnetic beads (Agencourt AMPure XP, Beckman Coulter). Pools were then subjected to 20 cycles of PCR using Phusion high-fidelity polymerase (Finnzymes). Single-strand products were obtained using Dynabeads® (Life Technologies) then PCR-amplified for 14 cycles with Phusion polymerase to incorporate the Illumina bridge amplification sequence. Final PCR products were purified and then quantified using PicoGreen® fluorescent dye (Quant-iT™ dsDNA Broad Range (BR) kit, Life Technologies). Final PCR products were diluted to 10 nM. Quality assessment was performed by the Agilent 2100 Bioanalyzer (Agilent Technologies). The template was sequenced on an Illumina GAIIx (Illumina) using standard Illumina protocols. Single-end sequencing was carried out for 38 cycles.

Base calling was performed using Illumina's Real Time Analysis (RTA) software, and sequence text files were generated using GERALD in Illumina's CASAVA v1.7 software package. Sequence files were then processed using a series of custom perl and python scripts. This data processing pipeline included culling of sequences that did not contain the Fsel partial restriction site and the specific, individual barcode identifier, sorting of bar-coded sequences and compression of duplicate reads. Sequences that matched more than one region of the reference sorghum genome, Sb1.4, were culled as well to prevent complications in placement and order in the genetic map. Polymorphisms between parental lines BTx623 and SC748-5 were identified and scored through the progeny as described by Morishige et al. (BMC Genomics 14:448, 2013). Genetic map construction was performed using JoinMap linkage mapping software (Van Ooijen and Voorrips, Joinmap® 3.0, software for the calculation of genetic linkage maps, Plant Research International, Wageningen, the Netherlands, 2001).

### Example 2

### Phenotyping

The RIL population was screened for reaction to anthracnose in multiple environments: College Station, Tex. in 2010, 2011, and 2012, and Tifton, Ga. in 2012. Trials in College Station were planted at the Texas A&M University Research Farm, and in Tifton, the trial was planted at the University of Georgia College of Agricultural and Environmental Sciences campus. Production practices standard for sorghum fertilization, irrigation, and pest management were used in each environment. In both locations, a randomized complete block design with two replications was used, and a plot consisted of a single row 5.2 m long with row spacing of 0.8 m. All test plots at the Texas A&M University Research Farm were inoculated with the anthracnose pathogen to ensure uniform disease pressure, whereas in Tifton, Ga., anthracnose development occurred via natural infection with the addition of overhead sprinkler irrigation to encourage disease spread.

For manual inoculation of anthracnose, a mixed inoculum of multiple Colletotrichum sublineolum strains was applied to ˜60 day old plants using colonized sorghum seed (Erpelding and Prom, Plant Pathology J. 5:28-34, 2006). The C. sublineolum isolates used in 2010 and 2011 were: AMP 119, AMP 123, AMP 129, AMP 132, AMP 134, AMP 150, and AMP 159. In 2012, a mixture of isolates FSP 2, FSP 5, FSP 7, FSP 35, FSP 36, FSP 44, FSP 50, and FSP 53 were used. A mixture of isolates from several susceptible lines was used as a source of inoculum in resistance screening to test multiple strains of the pathogen and eliminate confounding environmental effects. Additional disease pressure was provided by inoculated border rows of anthracnose-susceptible BTx623. Seasonal rainfall and humidity maintained disease pressure.

Anthracnose disease ratings were not made until acervuli (which are indicative of anthracnose) were detectable in susceptible checks. At that time, anthracnose disease incidence and severity ratings were taken approximately 2-4 times post anthesis and maturity. As appropriate, ratings were taken for leaf, head, and stalk infection, as well as whole plot infection. The rating scale is described in Table 1. The final whole plot disease rating was used in subsequent QTL analysis. Plants were also measured for height and days to flowering. Height measurements were taken concurrently with scoring for anthracnose. Maturity was determined by the date on which fifty percent of the plants in a plot were at 50% anthesis.

TABLE 1
Disease rating criteria used for anthracnose field phenotyping of sorghum
BTx623 × SC748-5 RIL population and parents.

| Rating | Description |
|---|---|
| 0 | No evaluation possible |
| 1 | Resistant - disease inconspicuous or present on an occasional plant, only |
| | specking occurs |
| 2 | Disease is present and up to 50% prevalence on all plants with low severity on |
| | each plant; apparently causing little damage |
| 3 | Disease is present and over 50% prevalence on all plants with low severity on |
| | each plant; apparently causing little damage |
| 4 | Disease is present with 100% prevalence on all plants; some severity which |
| | apparently is causing some damage |
| 5 | Disease is severe with 100% prevalence on all plants; estimated leaf area |
| | destroyed up to 25%; disease appears to be of economic importance |
| 6 | Disease is severe with 100% prevalence on all plants; estimated leaf area |
| | destroyed is between 25 to 50%; disease is of economic importance |
| 7 | Disease is severe with 100% prevalence on all plants; estimated leaf area |
| | destroyed is between 50 to 75%; disease is of economic importance |
| 8 | Disease is severe with 100% prevalence on all plants; estimated leaf area |
| | destroyed is above 75%; disease is of economic importance |
| 9 | Disease is severe with 100% prevalence on all plants; leaf area destroyed is |
| | 100%; death of leaves or plants due to disease |

Anthracnose infection ratings varied among both genotypes and environments,e primarily due to variation in the environments (Table 2). For example, in drought conditions in College Station, Tex. in 2011, anthracnose-susceptible BTx623 had an average score of 4.8, whereas the average ratings in 2010 and 2012 were 6.6 and 8.8, respectively, when greater rainfall occurred. Across all environments, the average disease score for SC748-5 was consistently 1 (resistant), which confirmed the stable resistance conferred by this genotype. Among the 117 RIL F₅ lines, there was a wide range of disease severity, which was variable upon annual environmental conditions (Table 2).

TABLE 2
Average observed phenotypic values for sorghum lines BTx623 and
SC748-5 and F₅ RIL progeny. Range of scored phenotypes is provided for
F₅ RIL progeny. Standard deviation is shown in parentheses next to
mean values. Heritability was calculated for each trait in each environment.

| | | | | | Anthracnose |
|---|---|---|---|---|---|
| | | | Maturity | Height (HT), | (AN), 1-9 |
| Environment | Genotype | | (MA), days | cm | rating |
| College Station, TX | BTx623 | | 78.2 | 132.84 | 6.6 |
| 2010 | SC748-5 | | 86 | 124.97 | 1 |
| | RILs | Mean | 80.8 (3.5) | 163.58 (35.81) | 3.9 (3.1) |
| | | (SD) | | | |
| | | Range | 73-87 | 85.09-256.54 | 1-9 |
| | | h² | 0.74 | 0.74 | 0.79 |
| College Station, TX | BTx623 | | 66 | 120.65 | 4.8 |
| 2011 | SC748-5 | | 73.5 | 105.66 | 1 |
| | RILs | Mean | 68.27 (3.51) | 138.07 (33.50) | 1.35 (0.79) |
| | | (SD) | | | |
| | RILs | Range | 60-79 | 63.5-233.68 | 1-5 |
| | | h² | 0.77 | 0.81 | 0.82 |
| College Station, TX | BTx623 | | 72 | 127.76 | 8.83 |
| 2012 | SC748-5 | | 77.5 | 107.11 | 1 |
| | RILs | Mean | 73.36 (5.11) | 157.10 (41.33) | 4.85 (3.28) |
| | | (SD) | | | |
| | RILs | Range | 60-86 | 114.3-144.78 | 1-9 |
| | | h² | 0.76 | 0.96 | 0.94 |
| Tifton, GA | BTx623 | | 75.25 | 134.62 | 8.8 |
| 2012 | SC748-5 | | 70 | 126.26 | 1 |
| | RILs | Mean | 68.65 (9.04) | 172.26 (35.56) | 5.93 (3.49) |
| | | (SD) | | | |
| | RILs | Range | 53-94 | 88.9-241.3 | 1-9 |
| | | h² | —^{†} | 0.94 | 0.96 |
| Combined Env. | | h² | 0.85 | 0.95 | 0.59 |

^{†}Heritability was not calculated due to too many missing data points.

### Example 3

### QTL Analysis

The average anthracnose disease rating from the two replications from each environment/year for each progeny was used for composite interval mapping (CIM) QTL analysis with 1000 permutations in QTL Cartographer 2.5 software (Wang et al., Windows qtl cartographer 2.5, North Carolina State University, Raleigh, N.C., 2012). The phenotypic data was also subjected to inclusive composite interval mapping (ICIM) analysis with 1000 permutations in QGene software version 4.3.10 (Joehanes and Nelson, Bioinformatics 24:2788-2789, 2008; Li et al., Genetics 175:361-374, 2007). Log of odds (LOD) significance threshold was calculated using the method as previously described (van Ooijen, Heredity 83:613-624, 1999).

Because multi-year, multi-location phenotypic data were available, the heritability was estimated in each environment and combined (h²) on an entry mean basis for each estimate. Main effects of line, environment location, year, and replication within a location in a specific year were treated as random effects. The R statistical software package lme4 was employed to calculate variance components (Bates and Maechler, Lme4: Linear mixed-effects models using s4 classes. R package version 0.999375-32, 2009).

As calculated in QGene software by the method of van Ooijen, the minimum LOD score for declaring a QTL significant was 3.9 for all traits evaluated (van Ooijen, Heredity 83:613-624, 1999). A previously reported QTL for maturity (MA) was observed on sorghum chromosome one with significant LOD scores of 4.9 to 7.7, explaining up to 27% of the phenotypic variance based on R2 values produced by QGene software (Table 3; Hart et al., Theor. Appl. Gen. 103:1232-1242, 2001; Joehanes and Nelson, Bioinformatics 24:2788-2789, 2008; Menz et al., Plant Mol. Biol. 48:483-499, 2002). The height genes, Dw2 and Dw3, segregate in this population. Both BTx623 and SC748-5 are three-dwarf sorghum s with BTx623 having a dominant Dw2 allele, whereas SC748-5 is dominant at Dw3. The physical positions of these QTL correspond to previously reported regions in the sorghum genome (Morishige et al., BMC Genomics 14:448, 2013). At Dw2, on sorghum chromosome six, LOD scores were observed between 6.1 and 8.3, accounting for up to 26% of the phenotypic variance. On sorghum chromosome 7, where Dw3 resides, LOD scores ranged from 3.3 to 6.5, accounting for up to 27% of the phenotypic variance (Table 3). While the LOD scores for maturity and height QTL did not meet the criteria for significance (i.e., LOD >3.9) in all environments examined, the same QTL were detected regardless of environment.

For anthracnose, one statistically significant QTL was consistently observed in all environments at the distal end of sorghum chromosome 5 (Table 3). The phenotypic proportion of variance explained by this QTL was as high as 72%, calculated by the R2 values in both QTL Cartographer and QGene software. While the peak LOD scores at this QTL ranged between 23.19 and 3.6 across the different environments, the peak consistently remained in the same position on the distal end of sorghum chromosome 5. Also, the same QTL was consistently observed in this position for anthracnose resistance ratings on foliage, stems, and panicles.

TABLE 3
QTLs detected following analysis of 117 F₅ progeny of a cross between BTx623 and SC748-5.

| | | | 2 LOD Confidence | | | | | |
|---|---|---|---|---|---|---|---|---|
| | | OTL Interval^{†} | Interval^{‡} | | | | | |
| | | Markers and | Markers and | Peak Marker | Environment + | | | Additive |
| Trait | Chromosome | locations | locations | and Location | Year | LOD | R² Value | Value^{§} |
| Maturity | 1 | c1_B_998-c1_F_1749 | c1_F_1147-c1_F_1749 | c1_F_1152 | CS 2010 | 7.7 | 0.27 | −2.15 |
| | | [0.0-14.9 cM] | [4.5-12.7 cM] | [7.1 cM] | CS 2011 | 4.9 | 0.18 | −1.58 |
| | | [20.54-48.81 Mbp] | [25.19-48.81 Mbp] | [25.47 Mbp] | CS 2012 | 1.6 | 0.06 | −1.09 |
| | | | | | GA 2012 | N/A | N/A | N/A |
| Height | 6 | c6_B_1228-c6_F_1382 | c6_F_1250-c6_F_1382 | c6_F_1250 | CS 2010 | 7.1 | 0.22 | +6.80 |
| | | [51.8-70.9 cM] | [57.1-70.9 cM] | [60.0 cM] | CS 2011 | 8.3 | 0.26 | +6.54 |
| | | [42.16-46.18 Mbp] | [42.65-46.18 Mbp] | [42.65 Mbp] | CS 2012 | 1.3 | 0.04 | +2.21 |
| | | | | | GA 2012 | 2.1 | 0.08 | +4.22 |
| Height | 7 | c7_B_1830-c7_F_1849 | c7_B_1830-c7_F_1849 | c7_F_1835 | CS 2010 | 3.4 | 0.13 | −6.16 |
| | | [67.2-71.9 cM] | [67.2-71.9 cM] | [70.3 cM] | CS 2011 | 6.5 | 0.27 | −7.92 |
| | | [58.39-58.89 Mbp] | [58.39-58.89 Mbp] | [58.54 Mbp] | CS 2012 | 3.4 | 0.13 | −7.09 |
| | | | | | GA 2012 | 2.4 | 0.09 | −5.50 |
| Anthracnose | 5 | c5_F_1666-c5_B_1937 | c5_F_1888-c5_F_1893 | c5_F_1893 | CS 2010 | 14.3 | 0.40 | +1.14 |
| | | [7.5-53.2 cM] | [40.7-42.5 cM] | [42.5 cM] | CS 2011 | 3.6 | 0.12 | +0.24 |
| | | [53.80-62.15 Mbp] | [59.97-60.77 Mbp] | [60.77 Mbp] | CS 2012 | 23.19 | 0.72 | +1.76 |
| | | | | | GA 2012 | 6.3 | 0.24 | +1.90 |

Traits analyzed included maturity, height, and anthracnose.
“NA” indicates no QTL detected.
^{†}Defined as the interval above the 3.9 LOD significance threshold.
^{‡}Defined as the interval containing the peak marker ±2 LOD.
^{§}The additive value is the value contributed by the BTx623 allele.

Traits analyzed included maturity, height, and anthracnose. “N/A” indicates no QTL detected. ^{†}Defined as the interval above the 3.9 LOD significance threshold. ^{†}Defined as the interval containing the peak marker ±2 LOD. ^{§}The additive value is the value contributed by the BTx623 allele.

### Example 4

### Sequencing of SC748-5

SC748-5 seedlings were grown in a growth chamber (14 hr light/10 hr dark) and leaf tissue was collected after 14 days. Genomic DNA was isolated using the FastPrep Extraction kit and FastPrep instrument (MP Biomedicals) as described above. An Illumina TruSeq DNA library was prepared by the Texas AgriLife Genomics Core Facility and the library sequenced in one lane on a HiSeq 2000 (Illumina). For sequencing, 100-bp paired-end reads were collected and, following base calling using Illumina's RTA software, the sequences were uploaded to the CLC Genomics Workbench (CLC Bio). Duplicate reads were removed using the Remove Duplicate Reads feature within CLC Genomics Workbench version 4.5.2 and the remaining reads trimmed using the Trim Sequences feature. The trimmed SC748-5 paired reads were mapped to the BTx623 reference genome (version Sbicolor-79; Paterson et al. Nature 457:551-556, 2009) and variants detected using the Map Reads to Reference and Quality-based Variant Detection features within the CLC Genomics Workbench, respectively. For read mapping, mismatch cost was set to 2, and insertion and deletion costs set to 3. Reads were required to align for at least 50% of their length, with similarity higher than 90% and non-specific read matches were mapped randomly. For Quality-based Variant Detection, the neighborhood radius was set to 5, minimum neighborhood quality set to 15, and minimum quality of the variant set to 20. Additionally, the maximum gap and mismatch count were set to 3, non-specific matches as well as broken pairs were ignored, the variant had to be present in both forward and reverse reads, and the minimum coverage for a variant call was set to 12. The minimum variant frequency was set to 35% to call heterozygotes. Annotations for the sorghum genome were downloaded from Phytozome (version Sbi 1.4), and were used to determine the presence of coding variants in SC748-5 as compared to BTx623. Orthologs of annotated genes in Arabidopsis were identified using the Rice Genome Annotation Project (Kawahara et al., Rice 6:1-10, 2013).

A total of 595,146,260 paired reads were produced by the 100 base paired-end sequencing run of the Illumina TruSeq library of SC748-5. Following duplicate read removal and quality trimming, a total of 522,995,084 paired reads remained for mapping against the BTx623 reference genome. The number of reads mapping to each of the ten chromosomes, the number of variants between the two genotypes and the average sequencing depth at variant sites are shown in Table 4. The numerous reads allowed observation of high-resolution, high-confidence polymorphisms in coding regions of interest on the distal end of sorghum chromosome 5, where the major QTL for anthracnose resistance is located (53.80 to 66.15 Mbp). Fifty seven potential candidate genes associated with disease resistance in plants based on gene ontology data from annotated plant species are located beneath the QTL interval where the LOD score exceeded the threshold for significance (LOD=3.9), (Table 5). The sequences generated in this study have been deposited in the NCBI Biosample database under accession numbers SAMN02688210 and SAMN02688211.

TABLE 4
Summary of mapping coverage and variants detected following
mapping and variant detection of SC748-5 sequence reads to
the BTx623 reference genome.

| | Number of Variant Sites | | Average Depth of |
|---|---|---|---|
| Sorghum | between BTx623 and | Total Read | Coverage at |
| Chromosome | SC748-5 | Count | Variant Sites |
| 1 | 206701 | 11121712 | 53.8 |
| 2 | 90717 | 5771769 | 63.6 |
| 3 | 167930 | 9320274 | 55.5 |
| 4 | 199940 | 10187119 | 50.9 |
| 5 | 143031 | 8017712 | 56 |
| 6 | 88968 | 5393078 | 60.6 |
| 7 | 108577 | 5657484 | 54.8 |
| 8 | 186273 | 9840499 | 52.8 |
| 9 | 95358 | 5514066 | 57.8 |
| 10 | 197328 | 10026787 | 50.8 |

The cost-effective, high-throughput sequencing of SC748-5 provided invaluable genetic information enabling the identification of significant sequence-level polymorphisms between SC748-5 and BTx623 in the numerous disease resistance-associated genes in the region of the QTL on chromosome 5 (Tables 5 and 6). Table 6 provides SNP markers that may be used in PCR-based assays to monitor introgression events. Of particular significance are variants in coding regions, particularly those that lead to amino acid changes. Within the most statistically significant QTL region on sorghum chromosome 5 (59.97-60.77 Mbp), five known gene families associated with plant disease resistance were observed. Each gene product exhibits a different mechanism of plant disease resistance, and any, or all, of these genes could be responsible for the resistance to anthracnose observed in SC748-5.

Genes Sb05g026250 (60044232-60046143 bp) and Sb05g026260 (600067359-60069221 bp) are SCARECROW-LIKE 14 transcription factor orthologs, which are known to play a role in xenobiotic stress responses (Fode et al., Plant Cell Online. 20:3122-3135, 2008; Ramel et al., J. Exp. Bot. 63:3999-4014, 2012). When compared to BTx623, SC748-5 had seven SNP/INDEL variants and five amino acid changes in Sb05g026250, whereas 25 SNP/INDEL variants and 18 amino acid changes were detected in Sb05g026260.

Two NBS-LRR genes were also observed in the region. The NBS-LRR genes are the classic R resistance genes. Often called Nibblers, these genes have a specific binding site to recognize a specific pathogen protein in the attacking fungus (Biruma et al., Theor. Appl. Gen. 124:1005-1015, 2012; Pan et al., J. Mol. Evol. 50:203-213, 2000). The recognition facilitates a signal kinase cascade involving many genes to deploy compounds such as phenolics and salicylic acid to the site of infection where the invading pathogen is cordoned off from the rest of the plant in a cell that is killed by the host, using primarily callose in addition to other wound response proteins to contain and kill the infecting agent. NBS-LRR gene Sb05g026470 (60360741-60364202 bp) contained 110 SNP/INDEL variants and eight amino acid changes in SC748-5 as compared to BTx623 and in the adjacent NBS-LRR gene, Sb05g026480 (60372348-60375425 bp), 228 SNP/INDEL variants and 118 amino acid changes were found.

Sb05g026490 (60376700-60377964 bp) encodes a glutathione-S-transferase, the product of which is stimulated by oxidative burst signals to assist in the catabolic break down of toxins (Chi et al., DNA Research 18:1-16, 2011; Levine et al., Cell 79:583-593, 1994; Salzman et al., Plant Physiol. 138:352-368, 2005). Twenty-six SNP/INDEL variants and 1 amino acid change in this gene were identified from SC748-5.

Sb05g026540 (60474285 to 60476244 bp) is located directly under the highest peak of the QTL and this gene encodes a critical enzyme in the phenylpropanoid pathway, flavonone-3-hydroxylase, known in other plants to play a role in biotic and abiotic disease resistance (Cheng et al., PLoS ONE 8:e54154, 2013; Cho et al., Physiol. Mol. Plant Pathol, 2005; Davies, Plant Physiol. 103:291, 1993). The flavonoid compounds associated with sorghum's response to anthracnose include luteolinidin, 5-methoxyluteolinidin and 3-deoxyanthocyanidin (Ibraheem et al., Genetics 184:915-926, 2010; Lo et al., Physiol. Mol. Plant Pathol. 55:263-273, 1999).

The final gene under the QTL for anthracnose resistance, Sb05g026570 (60526640 to 60528039 bp), is annotated as a putative R gene for disease resistance. Its function is defined as a receptor-like protein kinase that plays a role in the regulation of the defense response as part of the systemic acquired resistance process (Qi et al., Mol. Plant Pathol. 12:702-708, 2011). The sequence of gene Sb05g026570 in SC748-5 is divergent from BTx623 by 3 SNPs and 1 amino acid change.

TABLE 5
Sorghum genes annotated under the major anthracnose QTL on sorghum chromosome
5. Genes shown are those that lie underneath the QTL interval where the LOD score exceeded
the 3.9 LOD significance threshold. Amino acid changes in genes associated with plant disease
resistance were detected using resequencing data of parent SC748-5 in comparison to parent
BTx623. Arabidopsis orthologs were obtained from the Rice Genome Annotation Project.

| | AA | Start | | | Arabidopsis |
|---|---|---|---|---|---|
| Gene | Changes | Position | Stop Position | Biological Process | Ortholog |
| Sb05g022160 | 3 | 53814166 | 53815489 | defense response to fungus | AT5G43590 |
| Sb05g022230 | 4 | 53883773 | 53888305 | salicylic acid biosynthesis, | AT5G22000 |
| | | | | systematic acquired resistance | |
| Sb05g022500 | | 54441250 | 54442464 | wound response | AT5G13930 |
| Sb05g022510 | | 54452544 | 54453758 | wound response | AT5G13930 |
| Sb05g022800 | | 55112819 | 55114699 | defense response, apoptosis | AT1G58410 |
| Sb05g022940 | | 55294132 | 55295938 | defense response to fungus | AT3G04720 |
| Sb05g022950 | | 55302124 | 55302693 | defense response to fungus | AT3G04720 |
| Sb05g022960 | | 55307197 | 55308168 | defense response to fungus | AT3G04720 |
| Sb05g023580 | | 56306811 | 56311240 | defense response, apoptosis | AT3G50950 |
| Sb05g023690 | | 56425879 | 56427007 | wound response | AT5G24090 |
| Sb05g023700 | | 56434322 | 56435584 | wound response | AT5G24090 |
| Sb05g023710 | | 56447566 | 56448691 | wound response | AT5G24090 |
| Sb05g024020 | | 56929808 | 56933159 | defense response, apoptosis | AT3G14470 |
| Sb05g024030 | | 56937190 | 56942639 | defense response, apoptosis | AT3G46730 |
| Sb05g024126 | | 57066642 | 57069341 | defense response, apoptosis | AT3G46710 |
| Sb05g024200 | | 57225660 | 57227381 | salicylic acid biosynthesis, | AT3G14470 |
| | | | | systematic acquired resistance | |
| Sb05g024230 | | 57370289 | 57371722 | wound response, regulation of | AT5G48930 |
| | | | | flavonoid biosynthesis | |
| | | | | pathway | |
| Sb05g024240 | | 57379883 | 57386627 | defense response to fungus | AT1G15520 |
| Sb05g024380 | | 57568617 | 57570520 | defense response to fungus | AT2G26560 |
| Sb05g024880 | 1 | 58055848 | 58059255 | defense response, apoptosis | AT3G46730 |
| Sb05g024900 | | 58076916 | 58080591 | defense response, apoptosis | AT3G46730 |
| Sb05g024940 | | 58124482 | 58125223 | defense response | AT5G42510 |
| Sb05g025040 | 8 | 58184326 | 58186026 | cuticle development | AT5G43760 |
| Sb05g025190 | | 58333533 | 58337365 | defense response, apoptosis | AT3G46730 |
| Sb05g025440 | 2 | 58724313 | 58729677 | defense response, apoptosis | AT3G46730 |
| Sb05g025670 | | 59089807 | 59098658 | defense response, lignin | AT1G65870 |
| | | | | biosynthesis | |
| Sb05g025870 | 153 | 59350967 | 59354066 | defense response, apoptosis | AT3G46730 |
| Sb05g025880 | 32 | 59362285 | 59365186 | defense response, apoptosis | AT1G58400 |
| Sb05g026070 | 7 | 59692426 | 59693376 | defense response | AT5G42510 |
| Sb05g026250 | 5 | 60044232 | 60046143 | xenobiotic stress response | AT1G07530 |
| Sb05g026260 | 18 | 60067359 | 60069221 | xenobiotic stress response | AT1G07530 |
| Sb05g026470 | 50 | 60360741 | 60364202 | salicylic acid biosynthesis, | AT3G14470 |
| | | | | systematic acquired resistance | |
| Sb05g026480 | 118 | 60372348 | 60375425 | salicylic acid biosynthesis, | AT3G14470 |
| | | | | systematic acquired resistance | |
| Sb05g026490 | 1 | 60376700 | 60377964 | toxin catabolic process | AT1G10360 |
| Sb05g026540 | 1 | 60474285 | 60476244 | encodes a flavonone-3- | AT5G20400 |
| | | | | hydroxylase protein | |
| Sb05g026570 | 1 | 60526640 | 60528039 | regulation of defense response, | AT4G08850 |
| | | | | systematic acquired resistance | |
| Sb05g026920 | | 60938221 | 60940458 | salicylic acid biosynthesis, | AT3G14470 |
| | | | | systematic acquired resistance | |
| Sb05g026930 | | 60963661 | 60965513 | defense response, apoptosis | AT1G50180 |
| Sb05g026965 | | 61009529 | 61010503 | defense response, apoptosis | AT1G50180 |
| Sb05g027000 | | 61082967 | 61094282 | defense response, apoptosis | AT3G46730 |
| Sb05g027090 | | 61216936 | 61222508 | defense response to fungus | AT1G21250 |
| Sb05g027260 | | 61490220 | 61499773 | defense response, apoptosis | AT1G50180 |
| Sb05g027270 | | 61505660 | 61506610 | defense response, apoptosis | AT1G50180 |
| Sb05g027280 | | 61515609 | 61524117 | defense response, apoptosis | AT3G46730 |
| Sb05g027290 | | 61529781 | 61530731 | defense response, apoptosis | AT1G50180 |
| Sb05g027300 | | 61569054 | 61572701 | defense response, apoptosis | AT3G46730 |
| Sb05g027310 | | 61588226 | 61589176 | defense response, apoptosis | AT1G50180 |
| Sb05g027320 | 3 | 61589927 | 61591939 | defense response, apoptosis | AT3G46730 |
| Sb05g027380 | | 61657960 | 61659130 | wound response | AT5G24090 |
| Sb05g027450 | 2 | 61814780 | 61815331 | defense response, response to | AT3G53600 |
| | | | | chitin | |
| Sb05g027465 | | 61831234 | 61832958 | response to fungus, regulation | AT1G19640 |
| | | | | of hypersensitivity response | |
| Sb05g027480 | | 61868780 | 61869562 | defense response | AT1G20030 |
| Sb05g027620 | | 62055349 | 62057361 | salicylic acid biosynthesis, | AT3G14470 |
| | | | | systematic acquired resistance | |
| Sb05g027630 | | 62073987 | 62075284 | defense response | AT3G50950 |
| Sb05g027740 | 1 | 62150295 | 62152965 | xenobiotic stress response | AT1G07530 |
| Sb05g027760 | 4 | 62162581 | 62164664 | defense response, response to | AT1G07520 |
| | | | | chitin | |

| Gene | Annotated Function | Citation in Literature |
|---|---|---|
| Sb05g022160 | patatin putative | (Yang et al., 2007) |
| Sb05g022230 | RHF2A (RING-H2 GROUP F2A); protein binding/zinc | (Vannini et al., 2006) |
| | ion binding | |
| Sb05g022500 | TT4 (TRANSPARENT TESTA 4); naringenin-chalcone | (Tohge et al., 2007) |
| | synthase | |
| Sb05g022510 | TT4 (TRANSPARENT TESTA 4); naringenin-chalcone | (Yu et al., 2005) |
| | synthase | |
| Sb05g022800 | disease resistance protein (CC-NBS-LRR class) putative | (Ibraheem et al., 2010) |
| Sb05g022940 | PR4 (PATHOGENESIS-RELATED 4); chitin binding | (Narusaka et al., 2004) |
| Sb05g022950 | PR4 (PATHOGENESIS-RELATED 4); chitin binding | (Biruma et al., 2012) |
| Sb05g022960 | PR4 (PATHOGENESIS-RELATED 4); chitin binding | |
| Sb05g023580 | disease resistance protein (CC-NBS-LRR class) putative | (Tan et al., 2007) |
| Sb05g023690 | acidic endochitinase (CHIB1) | (Lu et al., 2012) |
| Sb05g023700 | acidic endochitinase (CHIB1) | |
| Sb05g023710 | acidic endochitinase (CHIB1) | |
| Sb05g024020 | disease resistance protein (NBS-LRR class) putative | (Ashfield et al., 2004) |
| Sb05g024030 | disease resistance protein (CC-NBS class) putative | (Yang et al., 2006) |
| Sb05g024126 | disease resistance protein (CC-NBS-LRR class) putative | (Rose et al., 2004) |
| Sb05g024200 | disease resistance protein (NBS-LRR class) putative | (Nandety et al., 2013) |
| Sb05g024230 | HCT (HYDROXYCINNAMOYL-COA | (Hoffmann et al., 2003) |
| | SHIKIMATE/QUINATE HYDROXYCINNAMOYL | |
| | TRANSFERASE) | |
| Sb05g024240 | PDR12 (PLEIOTROPIC DRUG RESISTANCE 12); | (Gechev et al., 2004) |
| | ATPase coupled to transmembrane movement of | |
| | substances | |
| Sb05g024380 | PLA2A (PHOSPHOLIPASE A 2A); lipase/nutrient | (Rietz et al., 2004) |
| | reservoir | |
| Sb05g024880 | disease resistance protein (CC-NBS class) putative | (Vaid et al., 2012) |
| Sb05g024900 | disease resistance protein (CC-NBS class) putative | |
| Sb05g024940 | disease resistance-responsive family protein | (Ralph et al., 2007) |
| Sb05g025040 | KCS20 (3-KETOACYL-COA SYNTHASE 20); fatty acid | (Wang et al., 2004) |
| | elongase, stilbene synthase | |
| Sb05g025190 | disease resistance protein (CC-NBS class) putative | |
| Sb05g025440 | disease resistance protein (CC-NBS class) putative | |
| Sb05g025670 | disease resistance-responsive family protein | (Seo et al., 2007) |
| Sb05g025870 | disease resistance protein (CC-NBS class) putative | |
| Sb05g025880 | disease resistance protein (CC-NBS-LRR class) putative | (Mun et al., 2009) |
| Sb05g026070 | disease resistance-responsive family protein | |
| Sb05g026250 | SCL14 (SCARECROW-LIKE 14); transcription factor | (Fode et al., 2008) |
| Sb05g026260 | SCL14 (SCARECROW-LIKE 14); transcription factor | |
| Sb05g026470 | disease resistance protein (NBS-LRR class) putative | |
| Sb05g026480 | disease resistance protein (NBS-LRR class) putative | |
| Sb05g026490 | ATGSTU18 (GLUTATHIONE S-TRANSFERASE TAU | (Kuśnierczyk et al., |
| | 18); glutathione transferase | 2007) |
| Sb05g026540 | oxidoreductase 2OG-Fe(II) oxygenase family protein | (Davies, 1993) |
| Sb05g026570 | kinase | (Qi et al., 2011) |
| Sb05g026920 | disease resistance protein (NBS-LRR class) putative | |
| Sb05g026930 | disease resistance protein (CC-NBS-LRR class) putative | (Tan and Wu, 2012) |
| Sb05g026965 | disease resistance protein (CC-NBS-LRR class) putative | |
| Sb05g027000 | disease resistance protein (CC-NBS class) putative | |
| Sb05g027090 | WAK1 (CELL WALL-ASSOCIATED KINASE); kinase | (Peleg-Grossman et al., |
| | | 2012) |
| Sb05g027260 | disease resistance protein (CC-NBS-LRR class) putative | |
| Sb05g027270 | disease resistance protein (CC-NBS-LRR class) putative | |
| Sb05g027280 | disease resistance protein (CC-NBS class) putative | |
| Sb05g027290 | disease resistance protein (CC-NBS-LRR class) putative | |
| Sb05g027300 | disease resistance protein (CC-NBS class) putative | |
| Sb05g027310 | disease resistance protein (CC-NBS-LRR class) putative | |
| Sb05g027320 | disease resistance protein (CC-NBS class) putative | |
| Sb05g027380 | acidic endochitinase (CHIB1) | |
| Sb05g027450 | zinc finger (C2H2 type) family protein | (Wang et al., 2008) |
| Sb05g027465 | JMT (JASMONIC ACID CARBOXYL | |
| | METHYLTRANSFERASE); jasmonate O- | |
| | methyltransferase | |
| Sb05g027480 | pathogenesis-related thaumatin family protein | (El-kereamy et al., 2011) |
| Sb05g027620 | disease resistance protein (NBS-LRR class) putative | |
| Sb05g027630 | disease resistance protein (CC-NBS-LRR class) putative | |
| Sb05g027740 | SCL14 (SCARECROW-LIKE 14); transcription factor | |
| Sb05g027760 | scarecrow transcription factor family protein | (Libault et al., 2007) |

TABLE 6
SNP states of sorghum genotypes BTx623 and SC748-5 in
markers with significant LOD scores for the major anthracnose
QTL on chromosome 5.

| Chromosome | Marker Name | Physical Position | BTx623 | SC748-5 |
|---|---|---|---|---|
| 5 | c5_F_1870 | 59978057 | G | T |
| 5 | c5_F_1888 | 60471998 | G | C |
| 5 | c5_F_1893 | 60771765 | G | A |

### Example 5

### Genetic Heritability of Resistance to Anthracnose

Statistical analysis of 117 F₅ lines in two replications, two geographical locations in three annual growing seasons indicated the presence of significant genetic differences among the parental and progeny genotypes, as well as environmental factors due to location and growing season. Heritability estimates within an environment for anthracnose resistance ranged from 0.79 to 0.96 (Table 2). These heritabilities are similar to those reported herein for both maturity and height, which are considered highly heritable in sorghum (Table 2). In the combined analysis of all environments, heritability dropped to 0.59 for anthracnose resistance, primarily due to the presence of significant genotype×environment interactions. This underlies the importance of analyzing and interpreting data on a per environment basis in addition to a combined analysis.

### Example 6

### F5 Genetic Map and QTL Analysis

A total of 840 unique, polymorphic SNP markers were identified in this F₅ RIL population by Digital Genotyping (Morishige et al, BMC Genomics 14:448, 2013). A total of 619 non-redundant markers had unique, informative polymorphisms and were included in the genetic map. Markers not included in the genetic map included polymorphisms that were too physically close to one another to statistically place them on linkage groups using the JoinMap software. Additionally, redundant markers were defined as polymorphisms between BTx623 and SC748-5 that showed identical segregation among the progeny and thus would provide no additional information if included. The F₅ linkage map included 3 SSR markers in addition to the 619 SNP markers over thirteen linkage groups, corresponding to the ten sorghum chromosomes. Due to the utilization of methyl-sensitive restriction enzyme, Fsel, sequences in expressed gene regions were targeted, whereas repetitive, heterochromatic regions are not represented. As a result of low marker coverage around centromeres, the long and short arms of three sorghum chromosomes (1, 2, and 5) remained as separate linkage groups (FIG. 1a and 1b). Table 7 illustrates the marker coverage over the ten sorghum chromosomes. Average marker coverage was one marker per 2.22 cM, and the total map length of all linkage groups was 1269.9 cM.

TABLE 7
Marker coverage across the ten sorghum chromosomes
in the BTx623 × SC748-5 F5 RIL genetic map.

| | | | Avg. Distance |
|---|---|---|---|
| | | | between Markers |
| Chromosome | Length (cM) | Marker Number | (cM) |
| 1 | 161.7 | 114 | 1.4 |
| 2 | 123.8 | 53 | 2.4 |
| 3 | 145.7 | 83 | 1.8 |
| 4 | 135.5 | 46 | 3.0 |
| 5 | 117.4 | 49 | 2.5 |
| 6 | 136.4 | 60 | 2.3 |
| 7 | 115.7 | 66 | 1.8 |
| 8 | 115 | 50 | 2.4 |
| 9 | 95.7 | 48 | 2.0 |
| 10 | 123 | 49 | 2.6 |
| Total | 1269.9 | 619 | 2.22 |

1. A sorghum plant comprising in its genome at least one introgressed allele locus associated with anthracnose resistance wherein the locus is in or genetically linked to a genomic region defined by loci c5_F_1666 (SEQ ID NOs:1 and 2) and c5_B_1937 (SEQ ID NOs:3 and 4) on sorghum chromosome 5, or within 15 cM thereof, or a progeny plant therefrom.

2. The sorghum plant of claim 1, wherein the locus is in a genomic region flanked by:
loci c5_F_1666 (SEQ ID NOs:1 and 2) and c5_F_1893 (SEQ ID NOs:5 and 6) on sorghum chromosome 5;
c5_F_1893 (SEQ ID NOs:5 and 6) and c5_B_1937 (SEQ ID NOs:3 and 4) on sorghum chromosome 5; or
c5_F_1888 (SEQ ID NOs:13 and 14) and c5_F_1893 (SEQ ID NOs:5 and 6) on sorghum chromosome 5;
or within 15 cM thereof.

3. The method of claim 1, wherein said allele locus comprises at least one polymorphic nucleic acid selected from the group consisting of SEQ ID NOs:1-16.

4. The method of claim 1, wherein said introgressed allele locus is introgressed from sorghum genotype SC748-5.

5. A part of the sorghum plant of claim 1, further defined as pollen, an ovule, a leaf, an embryo, a root, a root tip, an anther, a flower, a fruit, a stem, a shoot, a seed, a protoplast, a cell, and a callus.

6. The part of the sorghum plant of claim 5, wherein the part is a seed.

7. A method of detecting in at least one sorghum plant a genotype associated with anthracnose resistance, the method comprising the step of: (i) detecting in at least one sorghum plant an allele of at least one polymorphic nucleic acid that is associated with anthracnose resistance, wherein the polymorphic nucleic acid is in or genetically linked to a genomic region flanked by loci c5_F_1666 (SEQ ID NOs:1 and 2) and c5_B_1937 (SEQ ID NOs:3 and 4) on sorghum chromosome 5, or within 15 cM thereof.

8. The method of claim 7, further comprising the step of: (ii) identifying at least one sorghum plant in which a genotype associated with anthracnose resistance has been detected and denoting that the sorghum plant comprises a genotype associated with anthracnose resistance.

9. The method of claim 8, further comprising the step of: (iii) selecting a denoted sorghum plant from a population of plants.

10. The method of claim 7, wherein the polymorphic nucleic acid is located in or genetically linked to a genomic region flanked by:
loci c5_F_1666 (SEQ ID NOs:1 and 2) and c5_F_1893 (SEQ ID NOs:5 and 6) on sorghum chromosome 5;
c5_F_1893 (SEQ ID NOs:5 and 6) and c5_B_1937 (SEQ ID NOs:3 and 4) on sorghum chromosome 5; or
c5_F_1888 (SEQ ID NOs:13 and 14) and c5_F_1893 (SEQ ID NOs:5 and 6) on sorghum chromosome 5;
or within 15 cM thereof.

11. The method of claim 7, wherein at least one of said polymorphic nucleic acid is selected from the group consisting of SEQ ID NOs:1-16.

12. The method of claim 7, wherein said allele is introgressed from sorghum genotype SC748-5.

13. The method of claim 7, wherein the polymorphic nucleic acid is located within 2 cM of c5_F_1893 (SEQ ID NOs:5 and 6.)

14. A plant produced from the method of claim 9.

15. A seed that produces the plant of claim 14.

16. A method for producing a sorghum plant that comprises in its genome at least one introgressed locus associated with anthracnose resistance, the method comprising:
(i) crossing a first sorghum plant lacking a locus associated with anthracnose resistance with a second sorghum plant comprising a locus associated with anthracnose resistance located in a genomic region defined by loci c5_F_1666 (SEQ ID NOs:1 and 2) and c5_B_1937 (SEQ ID NOs:3 and 4) on sorghum chromosome 5, or within 15 cM thereof;
(ii) detecting in progeny resulting from said crossing at least a first polymorphic nucleic acid in or genetically linked to said locus associated with anthracnose resistance; and
(iii) selecting a sorghum plant comprising said polymorphic locus and said locus associated with anthracnose resistance.

17. The method of claim 16, further comprising the step of:
(iv) crossing the sorghum plant of step (iii) with itself or another sorghum plant to produce a further generation.

18. The method of claim 17, wherein steps (iii) and (iv) are repeated from about 3 times to about 10 times.

19. The method of claim 16, wherein the polymorphic nucleic acid is located in or genetically linked to a genomic region flanked by:
loci c5_F_1666 (SEQ ID NOs:1 and 2) and c5_F_1893 (SEQ ID NOs:5 and 6) on sorghum chromosome 5;
c5_F_1893 (SEQ ID NOs:5 and 6) and c5_B_1937 (SEQ ID NOs:3 and 4) on sorghum chromosome 5; or
c5_F_1888 (SEQ ID NOs:13 and 14) and c5_F_1893 (SEQ ID NOs:5 and 6) on sorghum chromosome 5;
or within 15 cM thereof.

20. A sorghum plant produced by the method of claim 18, or a progeny plant therefrom that comprises the introgressed locus associated with anthracnose resistance.

21. A method of sorghum plant breeding, the method comprising the steps of:
(i) selecting at least a first sorghum plant comprising at least one allele of a polymorphic nucleic acid that is genetically linked to a QTL associated with anthracnose resistance, wherein the QTL maps to a position between
loci c5_F_1666 (SEQ ID NOs:1 and 2) and c5_B_1937 (SEQ ID NOs:3 and 4) on sorghum chromosome 5, or within 15 cM thereof; and
(ii) crossing the first sorghum plant with itself or a second sorghum plant to produce progeny sorghum plants comprising the QTL associated with anthracnose resistance.

22. The method of claim 20, wherein the QTL maps to a position between:
loci c5_F_1666 (SEQ ID NOs:1 and 2) and c5_F_1893 (SEQ ID NOs:5 and 6) on sorghum chromosome 5;
c5_F_1893 (SEQ ID NOs:5 and 6) and c5_B_1937 (SEQ ID NOs:3 and 4) on sorghum chromosome 5; or
c5_F_1888 (SEQ ID NOs:13 and 14) and c5_F_1893 (SEQ ID NOs:5 and 6) on sorghum chromosome 5;
or within 15 cM thereof.

23. The method of claim 21, wherein at least one of said polymorphic nucleic acid that is genetically linked to a QTL associated with anthracnose resistance is selected from the group consisting of SEQ ID NOs:1-16.

24. The method of claim 21, wherein at least one polymorphic nucleic acid that is genetically linked to a QTL associated with anthracnose resistance maps within 40 cM, 20 cM, 15 cM, 10 cM, 5 cM, or 1 cM of the QTL associated with anthracnose resistance.

25. A method of introgressing an allele into a sorghum plant, the method comprising:
(i) genotyping at least one sorghum plant in a population with respect to at least one polymorphic nucleic acid located in or genetically linked to a genomic region defined by loci c5_F_1666 (SEQ ID NOs:1 and 2) and c5_B_1937 (SEQ ID NOs:3 and 4) on sorghum chromosome 5, or within 15 cM thereof;
(ii) selecting from the population at least one sorghum plant comprising at least one allele associated with anthracnose resistance.

26. The method of claim 25, wherein the polymorphic nucleic acid is located in a genomic region flanked by:
loci c5_F_1666 (SEQ ID NOs:1 and 2) and c5_F_1893 (SEQ ID NOs:5 and 6) on sorghum chromosome 5;
c5_F_1893 (SEQ ID NOs:5 and 6) and c5_B_1937 (SEQ ID NOs:3 and 4) on sorghum chromosome 5; or
c5_F_1888 (SEQ ID NOs:13 and 14) and c5_F_1893 (SEQ ID NOs:5 and 6) on sorghum chromosome 5;
or within 15 cM thereof.

27. The method of claim 25, wherein at least one of said polymorphic nucleic acid is selected from the group consisting of SEQ ID NOs:1-16.

28. A sorghum plant obtained by the method of claim 25.

29. A method of producing biofuel comprising:
(a) obtaining the plant of claim 1 or a part thereof; and
(b) producing biofueld from said plant or part thereof.

30. Use of the plant according to claim 1 for making food, feed, or biofuel.
